(19) United States
(12) Patent Application Publication
(10) Pub. No.: US 2002/0127223 A1
(43) Pub. Date: Sep. 12, 2002

(54) Method for reducing side effects of a drug

(76) Inventor: Kenneth N. Matsumura, Berkeley, CA (US)

Correspondence Address: Kenneth N. Matsumura, #100, 2107 Dwight, Berkeley, CA, 94704, US

(21) Appl. No.: 09/906,322

(22) Filed: Jul. 13, 2001

Related U.S. Application Data
(63) Continuation-in-part of application No. 07/322,209, filed on Mar. 13, 1989, now abandoned.
Application No. 07/322,209 is a continuation-in-part of application No. 07/007,763, filed on Jan. 27, 1987, now abandoned.
Application No. 07/007,763 is a continuation-in-part of application No. 06/631,806, filed on Jul. 17, 1984, now abandoned.

(51) Int. Cl. A61K 39/395; A61K 31/00

(52) U.S. Cl. 424/130.1; 514/1

(57) ABSTRACT

Method for reducing side-effects of a drug caused by undesired effects of said drug upon body cells which are not the intended target of said drug comprising the preferential delivery of antidote for said drug to said body cells when said drug is used, said preferential delivery effected by attaching to said antidote antibody with affinity for said body cells.

This is a continuation-in-part of application Ser. No. 07/322,209 filed Mar. 13, 1989, which in turn was a continuation-in-part of application Ser. No. 07/007,763 filed Jan. 27, 1986, which was a continuation-in-part of application Ser. No. 06/631,806 filed Jul. 17, 1984, all said preceeding applications having been abandoned.

### FIELD OF INVENTION

This invention is in the field of chemotherapy. More specifically, it deals with methods of reducing side effects of chemotherapeutic agents.

### BACKGROUND OF INVENTION

Drugs could be made more effective if side effects did not prevent their use in stronger dosages. Side effects are caused by their action not being sufficiently focussed on the body cells or microorganisms needing to be treated. The side effects are specifically caused by misdirected action of said drug on certain body cells which are not the intended target of said drug. For example, if the drug intended for other targets is nevertheless toxic to gastrointestinal cells, then nausea, vomiting, and diarrhea, as well as gastrointestinal bleeding can result. If the drug is toxic to hematopoietic (blood making) cells, anemia, susceptibility to infection (from insufficient number of white corpuscles), and bleeding can result. If the drug is toxic to cells of the skin, hair loss and rash can result.

The reason cancer is so difficult to eradicate from the body is that agents (hereinafter referred to as oncolytics or anti-cancer drugs) which kill cancer cells also kill normal dividing cells like those of the intestine, hematopoietic bone marrow, and the skin. If normal cells can be protected from the cytotoxic effects of anti-cancer agents, it would become safe to use higher doses of the anti-cancer agents. Since higher doses kill a higher percentage of cancer cells, it would become possible to kill those few cancer cells able to survive the lower dosages currently in use.

The reason viruses are so difficult to eradicate from the body is that agents which kill viruses also kill normal dividing cells. If normal cells can be protected from the cytotoxic effects of anti-viral agents, we would be able to use sufficient dosages of such agents to eradicate life-threatening viruses. For example, azidothymidine (AZT) has been found to be life-prolonging in the treatment of Acquired Immune Deficiency Syndrome (AIDS). According to Backgrounder (published by the National Cancer Institute, Office of Cancer Communications), Sep. 19, 1986, “AZT is a derivative of thymidine, one of the normal components of DNA (genes), which is needed by the host cell and the virus to make chemicals necessary for replication. When AZT enters a cell infected by HTLV-III (the virus that causes AIDS)), the drug floods the cell with false DNA building blocks so the virus cannot make copies of itself. Once this happens, viral infection and replication is halted, thereby protecting target cells.” Unfortunately, azidothymidine injected into the blood stream also reaches hematopoietic cells of bone marrow. There, azidothymidine halts replication of reproducing blood cells. The result is anemia and reduced white blood cell count. The NCI Backgrounder (just cited) states, “at high doses, it is likely that bone marrow suppression will be a limiting factor for the drug.” If we can protect these bone marrow cells from the harmful action of AZT, we would be able to use a higher dosage of AZT possibly leading to better treatments. While there are now substantial questions whether AIDS-associated virus is the cause of AIDS, the foregoing description would remain valid for the treatment of viral diseases in general where protection of normal dividing cells allows higher, more effective dosages of anti-viral agents.

Other anti-microbial drugs have serious side effects that limit their use. Gentamycin and related drugs like tobramycin and amikacin are, next to penicillin, some of the most important antibiotics in the treatment of sepsis in debilitated patients (e.g., the post-surgical, the elderly, the immunity-compromised). These drugs, however, also damage kidney cells as well as vestibular and auditory sensory cells and can not often be used in adequate dosages or for sufficient duration. Protecting the kidney, vestibular and auditory sensory cells from the harmful action of these antibiotics can enable more prolonged use of these drugs that can lead to saving of more lives.

### OBJECTS & SUMMARY OF THE INVENTION

I have invented a novel method for focussing of drug effect and reducing side effects of a drug by protecting body cells not meant to be affected by said drug from the unwanted effects of said drug. More specifically, my method is to preferentially deliver to the body cells needing said protection an antidote for said drug when said drug is used, said preferential delivery being accomplished, in one embodiment, by binding antibodies or antibody-comparables with affinity for said body cells (1) directly to said antidote or (2) to carriers such as proteins and other polymers carrying molecules of said antidote, or (3) to microbody carriers such as liposomes, erythrocytic ghosts, colloidal carriers, three dimensional dendrimers, or protenoid microspheres holding said antidote, said delivery being accomplished so as to enable said antidote to counteract undesirable actions of said drug on said body cells needing protection and to do so on said body cells needing protection in preference over the intended target of said drug.

In another embodiment, my method is to preferentially deliver to the body cells needing said protection an antidote for said drug when said drug is used, said preferential delivery being accomplished, in this other embodiment, by placing said antidote on a carrier having inherent affinity to be taken up by said cells needing protection, said affinity for uptake being greater by said cells needing protection than by the intended target of said drug, said uptake enabling said antidote to counteract undesirable actions of said drug on said body cells needing protection.

### DETAILED DESCRIPTION OF THE INVENTION

By the term “drug”, I mean chemotherapeutic agents such as those listed in The Pharmacologic Basis of Therapeutics (A. G. Gilman, L. S. Goodman, A. Gilman, editors. MacMillan Publishing Co. New York. 1980 and later editions). For this invention, the term “drug” includes ionizing radiation when used in treatment of diseases since the radioactive high energy particles act as drugs.

By the term “antidote”, I mean a chemical that counteracts the cellular effect of a specific drug. For example, folinic acid is an antidote for anti-cancer drug methotrexate, thymidine is an antidote for anti-cancer drug floxuridine (FUdR), oxypurinol is an antidote for 5-fluorouracil (5-FU), uridine is an antidote for azaribine, thiol containing chemicals such as thiourea, methionine (Burchenel, J. H. et al. Biochimie 60: 961-965. 1978; DiRe, F. et al. Cancer Chemother. Pharm. 25: 355-360. 1990) are antidotes for platinum and platinum derivatives, and deoxycytidine is an antidote for cytosine arabinoside. Folinic acid is a weak antidote for vinblastine and vincristine, but glutamic and aspartic acids, sodium glutamate and tryptophan are antidotes for vinblastine.

There is a certain logic about determining what chemical would serve as an antidote for any given drug:

1) If the drug acts by being a non-functional analog of a natural metabolite (for example,the enzyme dihydrofolate reductase is blocked by methotrexate, a non-functional analog of folinic acid, in the production of thymidine), one antidote for such a drug is the natural (functional) metabolite for which this drug is the analog (for methotrexate, folinic acid, which can compete for the enzyme binding site to prevent methotrexate from binding and blocking the enzyme).

2) If the drug acts by reversibly blocking an enzyme critically needed for the production of a specific metabolite (for example, the enzyme dihydrofolate reductase is blocked by methotrexate in the production of thymidine), one antidote for such a drug is the specific metabolite whose production is being blocked by the drug (for example, in the case of methotrexate, thymidine);

3) If natural resistance to a given drug is found to develop in cells, one can learn how cells become resistant and find a chemical which can make the cell resistant to the drug in the same manner; such a chemical could be an antidote. For example, cells often become more resistant to the action of a drug by increasing the synthesis of an enzyme that can help degrade the drug once the drug enters the cell. Said enzyme is an antidote for the drug. As a corollary to this rule, an enzyme that can function otherwise harmlessly in the intracellular milieu which degrades the drug can be an antidote. Some cells are resistant to the action of bleomycin because of the higher intracellular concentration of bleomycin hydrolase. Resistance to cytosine arabinoside has also been attributed to cytidine deaminase. Resistance to important aminoglycoside antibiotics has been attributed to acetylases and aminoglycoside-inactivating enzymes. Xanthine oxidase found plentifully in the liver detoxifies 6-mercaptopurine and azathioprine. These enzymes, when delivered into the cytoplasm of cells needing protection, are antidotes for the drugs they detoxify.

4) For ionizing radiation as a drug, one can find antidotes among chemicals known to be “radioprotectors”, such as WR-2721 and WR-1065 (Walter Reed Army Institute of Research, Washington, D.C.).

5) A new approach I am still exploring is to deliver into the cellular cytoplasm an exogenous excess of the target structures to which the drug binds (cisplatin to deoxyribonucleic acid), intercalates with (doxorubicin, daunorubicin with deoxyribonucleic acid), or directly alters (alkylators with DNA and components like guanine). In this case, fragments of DNA (e.g., guanine-base rich DNA fragments, artificially synthesized) would be the antidote. Once inside the cytoplasm, such decoy DNA fragments could react with entering cytotoxic drug molecules rendering them harmless to the “real” functioning DNA of the cell.

6) Some drugs act via generation of free-radicals inside cells (for example, doxorubicin). In such cases, free-radical scavengers like alpha-tocopherol, coenzyme Q, and N-acyl dehydroalanines can serve as antidotes (Solaini, G. Biochem. Biophys. Res. Comm. 147 (2): 572-80. 1987 & its references; Pascoe, G. A. Archives Bioch. Biophys. 256(1): 159-166. 1987). Doxorubicin's use is limited by its cardiotoxicity which appears to be particularly caused by free-radical generation. Aforementioned scavengers can serve as antidotes to the heart muscle and other cells. Metallothionein (Webb, M. In: The chemistry, biochemistryand biology of cadmium. Webb, M., ed. Elsevier/North Holland, Amsterdam, p. 195. 1979) is a low molecular weight protein exhibiting a protective action against heavy metal toxicity, such as of platinum drugs and also appears to have protective effect on cells against free-radical forming drugs, such as doxorubicin, bleomycin, peplomycin, and irradiation. Metallothionein has also been reported as protective against some alkylating drugs. One can use metallothionein directly as an antidote, or use a chemical, such as bismuth subnitrate which pre-induces the synthesis within cells of metallothionein (Satoh, M, et al. Cancer Chemother. Pharmacol. 21: 176-178. 1988). It would of course be common sense, and certainly obvious to those skilled in the art, that if one uses a pre-inducer, one would optimize the protective effect if one delivers the pre-inducer in advance of the use of the cancer drug so that there would be time for the metallothionein to be formed in the cell to be protected before the cell is exposed to the cancer drug. However, since cancer drugs are typically applied to patients repeatedly over many weeks, one can begin the pre-inducer treatment around the same time as the cancer drug. At first, the cells to be protected are not optimally protected, but within a few days, said cells will be optimally protected.

When seeking antidotes, one can use known empirical evidence or conduct in vivo or in vitro tests. For example, one can determine if a substance qualifies as an “antidote” for the purposes of this invention as follows: one introduces the test “antidote” into a tissue culture of body cells needing protection. Then the drug for which the antidote is being tested is introduced into the tissue culture. One assesses the effect that the test “antidote” has on the action of the drug on the cells in tissue culture so that one can determine the acceptability of the test “antidote”. For some antidotes, like proteins and enzymes, one may need to provide means for the test antidote to get inside the cultured body cells (for example, by placing the enzymes inside liposomes (discussed below) and allowing the enzymes to enter the cytoplasm when the membranes of the liposomes and the cell fuse.

There are many ways one can “preferentially deliver (the antidote) to the body cells needing protection.” One can covalently bind the antidote to antibodies having affinity to the body cells needing protection. The state of the art continues to advance in methodology but I refer to Hurwitz, E., Cancer Res. 35: 1175-. 1975 who describes a successful linkage via periodate oxidation of the drug to be bound and reaction of the oxidized drug with the antibody followed by borohydride reduction of the reagents. Particularly useful technique is that developed by Cytogen Corporation (Princeton, N.J., USA—Rodwell, J. D. et al. Proc. Nat. Acad. Sci. 83: 2632-2636. 1986) which uses the oligosaccharides found in the constant region of the heavy chains of an antibody. Chemical or enzymic oxidation of oligosaccharides to aldehydes generates groups that react with compounds containing such functional groups as amines, hydrazines, hydrazides, and thiosemicarbazides. This site-specific modification yields relatively uniform antibody conjugates that have unimpaired antigen binding characteristics. These “linker” conjugates are also “conditionally stable,” i.e., the linkers release drugs in response to a so-called “second signal.” Drugs linked to the antibodies attach to the target cells and a second signal, such as a proteolytic enzyme breaks up the conjugates, releasing the drug into the extracellular space surrounding the target cells. In addition to enzymes (such as complement, plasmin, and elastase), linkers can also be made sensitive to pH changes, or radioisotopes, for example. The use of the “second signal” is not always necessary since a cell will take into the interior drugs that attach to its outer membrane.

Prior to the work of Cytogen Corporation, generally, it was felt difficult to directly bind drugs onto the antibody without causing the loss of function of the drug or the antibody. Therefore, many found it preferable to bind the drug first to a carrier and the carrier in turn bound to the antibody. In this manner, it is possible to bind more molecules of a drug to the antibody without causing the loss of function of the antibody. A successful example is that of M. Garnett, M. J. Embleton, E. Jacobs & R. W. Baldwin (Int. J. of Cancer 31: 661-670. 1983) who use albumin as a carrier. Reisfeld and his group lately has been using conjugation technique employing an acid-sensitive linker, cis-aconitic anhydride (Proc. Nat. Acad. Sci. 85: 1189-1193. 1988). Another interesting, though less disirable, technique is the use of polyglutamic acid as an intermediate carrier, as described by G. F. Rowland (Nature 255: 487-. 1975).

In a preferred embodiment and especially when using antidotes which are enzymes, the antidotes are first placed within a microbody carrier such as liposomes, erythrocytic ghosts, colloidal carriers, or protenoid microspheres, which can hold within their structure many molecules of antidote.

In the case of liposomes, they are spherules formed when phospholipids are allowed to swell in aqueous compartments. Within the lipid or aqueous phase of liposomes, lipid or water-soluble substances, respectively, can be entrapped. Several of physical properties of liposomes can be varied at will: size (radius) can be adjusted from about 12 nm for unilamellar liposomes to up to several microns for the multilamellar versions, and a negative or positive surface charge can be imposed by the incorporation of charged amphiles (Gregoriadis, G., Methods in Enzymology 44: 698-. 1976). Control of permeability to entrapped substances, and of stability is also feasible by the addition of a sterol or other lipids into the liposomal structure (Gregoriadis, G., reference just cited).

Preferably, the liposome should be unilamellar, about 350 to 600 angstroms in diameter and either neutral or positively charged. Many methods are well-known in the art for making suitable liposomes (Juliano, R. L., Stamp, D., Biochem. Biophys. Res. Comm. 63 (3): 651-658. 1975; also Ann. New York Acad. Sci. 308: 411-432. 1978, also Canad. J. Physio. Pharmacy 57 (5): 535-539. 1979, and Biochem. Pharmacol. 27: 21-27. 1978; Kimelberg, H. K., Cancer Res. 36: 2949-2957. 1976; work of Barbet, J., Machy, P., & L. Leserman, J. Supramolecular Structure and Cellular Biochemistry 16: 243-258. 1981; Gregoriadis, G. Nature 265: 407-411. 1977; Gregoriadis, G., New England J. Med. 295 (3): 704-710. 1976 and 295 (14): 765-770. 1976). Plentiful information is available in the research literature that describes how to vary the size, charge and content of the liposomes (Gregoriadis, G., Leathwood, P. D., & Ryman, B. E., Fed. Europ. Biochem. Soc. Letters 14: 95-99. 1971; Gregoriadis, G., Fed. Europ. Biochem. Soc. Trans. 2: 117-119. 1974; Gregoriadis, G. & Ryman, B. E., Europ. J. Biochem. 24: 485-491. 1972; Magee, E. E., Miller, O. V., Nature 235: 339-340. 1972; Kobayashi, T., Gann 66: 719-720. 1975; Gregoriadis, G. Biochem. Soc. Trans. 2: 117. 1974; Gregoriadis, G. Fed. Europ. Bioch. Soc. Letters 36 (3): 292-. 1973; Gregoriadis, G. & E. D. Neerunjun Biochem. Biophys. Res. Comm. 65: 537-544. 1975).

Another microbody carrier, the erythrocytic ghost, is a carrier vesicle formed from erythrocytes or similar materials (Ropars, C., et al., eds. Proc. of 2nd International Meeting on Red Blood Cells as Carriers for Drugs: Potential Therapeutic Applications in Advances in Biosciences. Vol. 67: 1-260. Pergamon Press, NY. 1987; Kruse, C. A., et al. Biotech. and Applied Biochem. 11: 571-580. 1989; Brearley, C. A., et al. J. Pharm. Pharmacol. 42: 297-301. 1990).

Colloidal carriers are polymer particles capable of carrying molecules of antidote (Koosha, F. et. al. Critical Reviews in Therapeutic Drug Carrier Systems 6 (2): 117-130, especially 123. 1989; Muller, R. H. In Colloidal Carriers for Controlled Drug Delivery ;and Targeting. R. H. pp 277-335. Muller, ed. Wissenshaftliche Verlagsgesellschaft, Stuttgart & CRC Press, Boca Raton, Fla. 1991).

Protenoid microspheres are hollow microspheres made of synthetic protein and are often the size of erythrocytes or smaller (Dr. Robert Rosen, Dalhousie University, Halifax, Nova Scotia; also patents of Clinical Technologies Associates, Inc. of Elmsford, N.Y.). Although currently the application of these spheres is predominantly for oral delivery into the blood stream of drugs, these carriers can be made to carry antidotes in the blood stream to various target cells.

Depending on the type, chemistry, and physical characteristics of the microbody carrier, placing the antidote in such microbody carrier can be advantageous in that some microbody carriers possess an inherent affinity to be taken up by certain body cells, such as the hematopoietic cells in the bone marrow. One reason for this affinity is that some blood and bone marrow cells have a natural tendency to “engulf” a particulate matter and a microbody carrier constitutes such a particulate matter. In such cases, one can preferentially deliver the antidote to the normal cells needing protection over the drug's intended target merely by designing, developing, or selecting the microbody carrier with the appropriate affinity.

For example, by modifying the structure and the chemical composition of the liposomes, I have discovered that one can direct the liposomes to different cells in the body, for example, the bone marrow cells or the liver cells. Since many drugs, especially oncolytics, affect bone marrow and liver cells detrimentally, one can take advantage of my discovery to deliver the antidotes to the aforementioned normal cells.

Through several years of intense experimentation, I have learned that certain types of liposomes have advantages over other types for the purposes of this invention. Liposomes can be made largely of lipids which are either “fluidy,” (liquid-crystalline state) or “solid” at the body temperature. I will hereinafter refer to the fluidy liposomes as being “soft-shelled,” and “solid” liposomes as being “hard-shelled,” as others have also done. Examples of soft-shelled liposomes are those made of egg phosphatidylcholine (<0) and brain phosphatidylserine (13), numbers in parenthesis being their solid to liquid-crystalline transition temperature in degrees Centigrade. Examples of hard-shelled liposomes are those made of distearoylphophatidylglycerol (53.7) and distearoylphosphatidylcholine (55.0), numbers in parenthesis again being their solid to liquid-crystalline transition temperatures which are all above the body temperature of 37-40 C. There are also lipids whose solid to liquid-crystalline transition temperatures are close to the body temperature such that liposomes made from them fall somewhere in between being soft and hard-shelled, and can be referred to as being “intermediate hard-shelled.” These lipids are: dipalmitoylphosphatidylglycerol (40.0) and dipalmitoylphosphatidylcholine (41.5). Most unexpectedly, I discovered that soft-shelled liposomes possess a greater inherent ability to deliver the antidote to bone marrow cells and counteract the drug toxicity when compared to hard-shelled liposomes. Intermediate hard-shelled liposomes were somewhere in between. Therefore, in one preferred embodiment, the antidote is placed within small, soft-shelled liposomes (of 120 to 950 Angstroms in diameter, made of egg phosphatidylcholine and cholesterol, in the molar ratio of 65:35, respectively) when the goal is to protect the bone marrow cells from toxic cancer drugs. Soft-shelled liposomes also possess greater ability to deliver the antidote to hepatocytes.

As for erythrocytic ghosts, these carriers also can have a natural affinity to be taken up by the reticuloendothelial cells, enabling the delivery of antidotes to such cells (Deloach, J. R. Med. Res. Rev. 6: 487-504. 1986). Colloidal carriers can be made to possess inherent tendency to deliver antidotes to bone marrow cells by down sizing (for example to no larger than 200 nm) and by manipulating the surface characteristics (Illum, L, et al. Life Sc. 40: 367-374. 1987).

Particulate carriers, such as ferritin can also have a tendency to be engulfed by certain bone marrow cells, including the macrophages, and the antidote can be bound to this type of carrier if a preferential delivery to bone marrow cells is desired. Particulate carriers themselves can also be bound to microbody carriers and other carriers carrying antidote for targeting to bone marrow cells. Dendritic dendrimers (Joe Alper. Science 251:1562-1564. 1991 including the references listed) are hyperbranching polymers as large as 100 Angstroms capable of carrying hundreds of antidotal chemicals. These dendrimers can also be engulfed by bone marrow cells.

Microbody carriers, such as liposomes, can be made to home in on target cells also by attaching antibody-comparables which include antibodies (Barbet, J., Machy, P., & L. Leserman, J. Supramolecular Structure and Cellular Biochemistry 16: 243-258. 1981 (Cellular Recognition, p. 237-252); Leserman, L., Barbet, J., & F. Kourilsky, Nature 288: 602-604. 1980; Machy, P., Pierres, M., Barbet, J., & L. Leserman, J. Immunology 129: 2098-2102. 1982; Machy, P., & L. Leserman, EMBO J. 3(9): 1971-1977. 1984; Konno, H. et al. Cancer Res. 47: 4471-4477, 1987), antibody “mimetics” (Saragovi, H. U., et al. Science 253: 792-794. 1991) and template-mediated synthesized polymers (Amato, I. Science 253: 1358. 1991; Dhal, P. K. & Arnold, F. H., Journal of American Chemical Society, Sep. 11, 1991). An antibody “mimetic” is a chemical based on the structure of an antibody. A template-mediated synthesized polymer is a chemical which is tailor-made to an antigen using the latter as a template. The use of antibodies and antibody-comparables provides a substantial advantage over other targeting methods because virtually any cell is targetable using antibodies and comparables.

Suitable antibodies for delivering the liposomes to target cells can be made by standard immunological methods, including that described by G. Gregoriadis (Biochem. Biophysic. Res. Comm. 65: 537-544. 1975). Monoclonal antibody production methods, however, is the best way to provide large amounts of purified antibodies (Kohler, G. & C. Milstein, Nature 256: 495-497. 1975; Barbet, J., Machy, P., & L Leserman, cited above). Preferably, the antibodies used in this invention should be non-complement-fixing. There are now many suitable commercially available antibodies (e.g., MRX OX1 mouse anti-rat leukocyte common antigen; OKT-10 mouse anti-human bone marrow cells). For the bone marrow stem cells and precursor cells of blood corpuscles and platelets, one can use antibodies comparable to those active against T200 glycoprotein of the mouse bone marrow cells. For the gastrointestinal cells, it would be preferable to use IGG₁ monoclonal antibodies against colon and small intestinal crypt and stem cells' laterobasal membrane. Other cells of the alimentary system needing protection includes oropharyngeal, esophageal, and gastric cells. For the skin cells, it would be preferable to use monoclonal IGG₁ antibodies against basal keratinocytes (and hair follicle keratinocytes). While some normal dividing cells divide less frequently and may not need protection when anti-mitotic cytoxic drugs are used, such cells may need protection under certain circumstances of drug use, such as in prolonged use or ultra-high dose. The previous sentence refers to cells including respiratory epithelial cells, urinary epithelial cells, and hepatocytes. Some drugs have specific organ toxicity (amikacin antibiotics to renal cells and otic cells; doxorubicin to cardiac muscle cells) and in such cases antibodies with affinity to the affected organs would be used.

Aside from antibodies and antibody comparables, one can bind to carriers a humoral factor which binds to a receptor on the surface of certain target cells and which also stimulates its own entry into the bound cell, without which ability one would need to provide for something like the “second signal” release (mentioned above) of the antidote off the carrier and the humoral factor. In this manner one can use substances such as the globulin transferrin which binds to a receptor on erythogenic cells which stimulates its own endocytosis into the erythrogenic cell. Of course, the humoral factor itself can be the carrier, but the use of another carrier provides for a considerable advantage in delivering a larger number of antidote molecules into cells.

The dosage of the antidote delivered to cells needing protection must be adequate to provide said protection. For example, in using folinic acid, one must deliver to the cell being protected at least one mole of folinic acid for each mole of methotrexate entering said cell. There may be advantages in injecting liposomes directly into the arteries feeding the cells needing protection (e.g., coeliac and superior mesenteric arteries feeding gastrointestinal cells).

The antidote should be introduced into the actively circulating body fluid (blood) bathing the cells needing protection before the cytotoxic drug is introduced into the same medium so as to allow the antidote time to reach its target cells. While an antidote like folinic acid has been found to attenuate the toxic effects of methotrexate even when administered 3 hours following methotrexate, attenuation is greater sooner one administers the antidote. In the case of this invention, the antidote preferably would be administered from two to twelve hours before the cytotoxic drug because it takes time for the antidote to be delivered to the cells needing protection because the antibody- or other delivery methods are relatively slow. Of course, one can hasten the delivery by increasing the amount of antibody- or carrier bound-antidote, but economic consideration could limit that approach. If a toxic drug is injected daily, liposomes should, of course, be also given daily, for as long as toxic levels of the drug remain in the blood stream. Even where the toxic drug is given only once, if the dosage is sufficiently high as to maintain a toxic level in the blood stream for a few days, it follows that it would be helpful to administer the liposomes on a daily basis.

One can make in advance of need antidote-carrier-antibody complexes (including antidote inside liposome bound to antibody). For example, any drug which has been found to be an antidote to another drug can be complexed with antibody to bone marrow stem cells or with antibody to colonic cells or with antibody to basal keratinocyte cells, etc. and each type of complex can be kept on cryogenic storage shelves for later use.

The following are examples of how to practice the invention. They are presented merely as an illustration and should not be construed to mean that the scope of the invention is limiting to the examples or even that the examples necessarily represent the best modes of operation.

### EXAMPLE

Unilamellar liposomes are made containing folinic acid (sodium salt) using egg phosphatidyl choline, cholesterol, and dipalmitoyl phosphatidyl ethanolamine 3-(2-pyridyldithio) propionate in molar ratio of 64:35:1 and to the liposomes are bound antibodies with affinity to bone marrow precursors of the white blood corpuscles) using a method adapted from Barbet (J. Supramol. Structure & Cell Biochem. previously cited). These liposomes are injected intravenously several hours prior to the administration of methotrexate for the treatment of cancer. Bone marrow toxicity of methotrexate is considerably reduced. It is particularly advantageous to simultaneously use 1) liposomes binding antibodies with affinity to gastrointestinal, esophageal, and oropharyngeal cells, 2) liposomes binding antibodies with affinity to cells of the skin, and 3) liposomes binding antibodies with affinity to the bone marrow stem cells, all the liposomes containing folinic acid or thymidine. Methotrexate can then be safely administered at a dosage easily two to three times that usually tolerated. In the case of liposomes containing thymidine, 5-FuDR and methotrexate together may be used.

### EXAMPLE 2

Unilamellar liposomes are made containing 2-deoxyguanosine and to the liposomes are bound antibodies with affinity to bone marrow cells, as in Example 1. These liposomes are injected intravenously several hours prior to the administration of gancyclovir for the treatment of CMV retinitis in AIDS patients. The liposomes are injected daily for as long as gancyclovir injections (twice daily) are continued. Bone marrow toxicity of gancyclovir is considerably reduced. The number of patients needing to withdraw from gancyclovir use due to bone marrow toxicity is substantially reduced and some patients are able to tolerate 25% higher doses of gancyclovir.

### EXAMPLE 3

As in Example 1 except liposomes incorporating cytidine deaminase instead of folinic acid and the drug is cytosine arabinoside instead of methotrexate.

### EXAMPLE 4

As in Example 1 except liposome incorporating bleomycin hydrolase instead of folinic acid and the drug is bleomycin instead of methotrexate.

### EXAMPLE 5

As in Example 1 except liposomes incorporating uridine instead of folinic acid and the drug is azaribine instead of methotrexate.

### EXAMPLE 6

As in Example 1 except liposome incorporating deoxycytidine instead of folinic acid and the drug is cytosine arabinoside instead of methotrexate.

### EXAMPLE 7

Instead of using liposomes as in above examples, the antidote is bound to antibodies using acid-sensitive linker, cis-aconitic anhydride. Otherwise, as in Examples 1, 2, 5, and 6.

Regarding the delivery of antidote via liposomes specially constructed for non-antibody targeting, the following steps illustrate the method. Small, unilamellar liposomes containing monosodium glutamate, size ranging from 120 to 950 Angstroms diameter, are made from egg phosphatidylcholine and cholesterol (14 mg and 3.87 mg, respectively) by evaporating the lipids dissolved in 2 ml of chloroform onto the inner surface of a 250 ml round-bottom flask, adding an aqueous L-buffer solution containing 32 mg/ml sodium glutamate, vortexing the mixture until lipids are uncoated from the flask, and then sonicating the cloudy mixture in bath type sonicator until clear. A volume of 0.5 ml of this suspension, representing 1.25 micromoles of lipid, sterilized by passage through a 0.22 micron bacteriological filter, is injected intravenously to a 100 gram rat 24 & 2 hours before and 24, 48, 72, and 96 hours after an intraperitoneal injection of vinblastine at the dose of 0.75 mg/kg. Although animals given only vinblastine experience weight loss (from gastrointestinal toxicity of anorexia and diarrhea) and substantial bone marrow destruction, animals given above described liposomes show much abatement of weight loss and marrow suppression.

Other examples include substituting the above drugs and their antidotes with other drug-antidote pairs mentioned in this specification.

A further example: my invention can also be used to create disease models in organisms by allowing a cytotoxic drug to kill or make sick certain body cells while protecting other body cells that can be affected by the cytotoxic agent. For example, one can use FUdR to damage colonic mucosal cells to simulate colitis while protecting small intestine, stomach, esophageal, oropharyngeal, bone marrow, and basal keratinocyte cells from the cytotoxic action of FUdR by the preferential delivery to the latter cells of thymidine using the methods described.

Having described the invention, I claim:

1. Method for reducing undesirable effect of a drug upon a body cell which is not the intended target of said drug, said drug having an antidote
capable of counteracting the cellular effect of said drug and
capable of being attached to antibody-comparable possessing affinity for said body cell substantially greater than for target of said drug, comprising:
attaching said antidote to said antibody-comparable,
introducing into body fluid that bathes said body cell in a mammalian organism said antidote attached to said antibody-comparable and delivering said antidote to said body cell so as to counteract said drug effect on said body cell in preference over said intended target of said drug when said drug is used in said organism while substantially ensuring said antidote from counteracting said drug except on said body cell.

2. Method for reducing undesired effect of a d rug upon a body cell which is not the intended target of said drug, said drug having an antidote
capable of counteracting the cellular effect of said drug and
capable of being carried by a carrier to which antibody-comparable with affinity for said body cell can be attached, comprising:
placing said antidote on said carrier to which said antibody-comparable is attached,
introducing into body fluid that bathes said body cell in a mammalian organism said antidote carried on said carrier to which said antibody-comparable is attached and delivering said antidote to said body cell so as to counteract said drug effect on said body cell in preference over said intended target of said drag when said drug is used in said organism while substantially ensuring said antidote from counteracting said drug except on said body cell.

3. Method for reducing undesired effect of a drug upon a body cell which is not the intended target of said drug, said drug having an antidote
capable of counteracting the cellular effect of said drug and
capable of being carried by a carrier possessing inherent affinity to be taken up by said body cell so as to allow said antidote to counteract said drug effect on said body cell, comprising:
placing said antidote on said carrier,
introducing into body fluid that bathes said body cell in a mammalian organism said antidote carried on said carrier and delivering said antidote to said body cell so as to counteract said drug effect on said body cell in preference over said intended target of said drug when said drug is used in said organism while substantially ensuring that said antidote not counteract said drug except on said body cell.

4. Method of claim 1 or 2 wherein said antibody-comparable is an antibody.

5. Method of claim 1 or 2 wherein said antibody-comparable is an antibodymimetic.

6. Method of claim 1 or 2 wherein said antibody comparable is a template-mediated polymer.

7. Method of claim 2, 3, 4, 5, or 6 wherein said carrier is a liposome.

8. Method of claim 2, 3, 4, 5 or 6 wherein said carrier is an erythrocytic ghost.

8. Method of claim 2, 3, 4, 5 or 6 wherein said carrier is a protenoid microsphere.

9. Method of claim 2, 3, 4, 5 or 6 wherein said carrier is a small, unilamellar liposome.

10. Method of claim 2, 3, 4, 5, or 6 wherein said carrier is a small, soft-shelled, unilamellar liposome.

11. Method of claim 2, 3, 4, 5, or 6 wherein said carrier is a small, unilamellar liposome made substantially of egg phosphatidylcholine and cholesterol in the molar ratio of about 65:35, respectively.

12. Method of claim 2, 3, 4, 5, or 6 wherein said carrier is a small, intermediate hard-shelled unilamellar liposome.

13. Method of claim 2, 3, 4, 5 or 6 wherein said carrier is a dendritic dendrimer.

14. Method of claim 2, 3, 4, or 5 wherein said undesired effect is granulocytopenia and said carrier employed is a liposome.

15. Method of 1, 2, 3, 4, 5, 6, 7, 8, 9, 10, 11, 12, or 13 wherein said antidote is glutamate.

16. Method of 1, 2, 3, 4, 5, 6, 7, 8, 9, 10, 11, 12, or 13 wherein said antidote is folinic acid.

17. Method of 1, 2, 3, 4, 5, 6, 7, 8, 9, 10, 11, 12, or 13 wherein said antidote is 2-deoxyguanosine.

18. Method of 1, 2, 3, 4, 5, 6, 7, 8, 9, 10, 11, 12, or 13 wherein said antidote is thymidine.

19. Method of 1, 2, 3, 4, 5, 6, 7, 8, 9, 10, 11, 12, or 13 wherein said antidote is deoxycytidine.

20. Method of 1, 2, 3, 4, 5, 6, 7, 8, 9, 10, 11, 12, or 13 wherein said antidote is oxypurinol.

21. Method of 1, 2, 3, 4, 5, 6, 7, 8, 9, 10, 11, 12, or 13 wherein said antidote is tryptophan.

22. Method of 1, 2, 3, 4, 5, 6, 7, 8, 9, 10, 11, 12, or 13 wherein said antidote is uridine.

23. Method of 1, 2, 3, 4, 5, 6, 7, 8, 9, 10, 11, 12, or 13 wherein said antidote is aspartic acid.

24. Method of 1, 2, 3, 4, 5, 6, 7, 8, 9, 10, 11, 12, or 13 wherein said antidote is bleomycin hydrolase.

25. Method of 1, 2, 3, 4, 5, 6, 7, 8, 9, 10, 11, 12, or 13 wherein said antidote is xanthine oxidase.

26. Method of 1, 2, 3, 4, 5, 6, 7, 8, 9, 10, 11, 12, or 13 wherein said antidote is a free radical scavenger.

27. Method of 1, 2, 3, 4, 5, 6, 7, 8, 9, 10, 11, 12, or 13 wherein said antidote is a fragment of deoxynucleic acid.

28. Method of 1, 2, 3, 4, 5, 6, 7, 8, 9, 10, 11, 12, or 13 wherein said antidote is a thiol-containing chemical capable of counteracting said drug effect of a platinum drug.

29. A small, unilamellar liposome holding metallothionein.

30. A small, unilamellar liposome holding a pre-inducer chemical for an antidote.

31. Claim 30 wherein the antidote is metallothionein.
